(19) United States
(12) Patent Application Publication
(10) Pub. No.: US 2022/0117466 A1
(43) Pub. Date: Apr. 21, 2022

(54) Endoscopes With Improved Compatibility To Oxidative Processings And Methods For Manufacturing And Repairing The Same

(71) Applicant: TSO3 Inc., Quebec (CA)

(72) Inventors: Jean-Luc Lemyre, Quebec City (CA); Bradley John Catalone, Hershey, PA (US); Sébastien Bouchard, Quebec City (CA)

(73) Assignee: TSO3 Inc., Quebec, QC (CA)

(21) Appl. No.: 17/298,356

(22) PCT Filed: Nov. 27, 2019
(86) PCT No.: PCT/IB2019/060253

§ 371 Date: May 28, 2021

Related U.S. Application Data
(60) Provisional application No. 62/773,874, filed on Nov. 30, 2018.

(51) Int. Cl. A61B 1/00; A61B 1/273; A61B 1/005; A61L 29/14; A61L 2/20

(52) U.S. Cl. CPC A61B 1/00078; A61B 1/0011; A61B 1/2736; A61B 1/0055; A61L 2202/24; A61L 2/202; A61L 2/208; A61L 2400/10; A61L 29/14

(57) ABSTRACT

An endoscope with particular adhesive elements and/or particular internal dry lubricants have improved compatibility to various oxidative processes such as the ones used for terminal sterilization. The particular adhesive elements and particular internal dry lubricants are disclosed herein. A method of manufacturing an endoscope and a method of repairing an old endoscope are also disclosed herein.

### CROSS-REFERENCE TO RELATED APPLICATIONS

This application claims the benefit of and priority to U.S. Provisional Patent Application Ser. No. 62/773,874, filed Nov. 30, 2018, which is incorporated herein by reference in its entirety.

### BACKGROUND

Gastro-Intestinal endoscopes (GIEs) currently are typically designed with materials compatible with high level disinfection, without consideration for terminal sterilization specificities. For this reason, some of the GIEs in use today have components that are not fully compatible with oxidative process sterilization. Depending on the model and manufacturer of the GIEs, these compatibility issues may affect longevity of the instrument.

Therefore, improvements in endoscopes and GIEs are desirable.

### BRIEF DESCRIPTION OF THE DRAWINGS

Advantages of the present disclosure will be readily appreciated as the same becomes better understood by reference to the following detailed description when considered in connection with the accompanying drawings.

FIG. 1 is a partial perspective view of an endoscope.

FIG. 2 is a partial cross-sectional view of an endoscope.

FIG. 3 is another partial cross-sectional view of an endoscope.

FIG. 4 is a cross-sectional view an endoscope.

FIG. 5 is a front plan view of an endoscope having a light lens and a camera objective.

FIG. 6 is a partial perspective view of an endoscope with a distal cap spaced from a distal end region.

FIG. 7 is a partial perspective view of an endoscope with the distal cap engaged with the distal end region.

FIG. 8 is a partial cross-sectional perspective view of an endoscope including a dry lubricant.

### DETAILED DESCRIPTION

Endoscopes are complex medical instruments with many parts and many different materials. Different types of adhesives are required at multiple locations to bond and seal these components. It is now known that when medical instruments present compatibility issues with sterilization or disinfection methods, the issues typically relate to the adhesives and/or internal lubricant. For example, these compatibility issues may cause the adhesive and/or internal lubricant of the endoscope to peel, crack, foam, lose adhesion, or otherwise deteriorate.

Endoscopes, particularly GIEs, often contain multiple internal components including channels, wires (e.g. guide wires), cables (e.g. guide cables, electronic cables, etc.) to angulate the instrument, and fiber optic bundles. For example, U.S. The back and forth movements of these internal components relative to one another require lubrication to prevent internal damage and to ease the operation of the endoscope. Currently fielded GIEs use molybdenum disulfide (MoS₂) for that purpose. Molybdenum disulfide, commonly referred as moly, is a well-known, dry lubricant used in various applications that require lubrication combined with a resistance to vacuum and/or high temperature. However, moly lubricant typically has low compatibility with hydrogen peroxide. During sterilization under vacuum, an endoscope vent port needs to be opened to relieve pressure differential and avoid endoscope damage. This is done with a simple pressure vent cap provided by the manufacturer, which allows sterilant vapors to come in contact with internal components of the instrument, allowing the sterilant to react with the moly lubricant having relatively low compatibility.

Insertion tubes may also show premature damage related to H₂O₂ sterilization. This can manifest by delamination of the clear outer polymer coat followed by different types of damage to the main polymer cover such as cracking, piercing or buckling.

The present disclosure proposes various distinct improvements that can be made to key sensitive components of endoscopes, and more particularly GIEs. As detailed below, materials having an improved compatibility with highly oxidative processing such as H₂O₂, O₃, H₂O₂+O₃, Plasma, H₂O₂+ Plasma, peracetic acid with terminal sterilization, liquid sterilization or high-level disinfection have been analyzed and specifically selected for specific needs. These improvements, alone or in combination, depending on a given application and as detailed below, can increase the lifetime of endoscopes. Tests have shown that a typical GIE generally withstands less than 20 sterilization cycles using H₂O₂ and Ozone as sterilant while a GIE modified according to the present disclosure typically withstands a greater number of cycles, greater than 20 in the preliminary tests. Thus, for the purpose of this disclosure, an endoscope having improved or enhanced compatibility with oxidative processing is an endoscope capable of withstanding greater than 20, 25, or 30 sterilizations cycles using H₂O₂, Ozone, or a combination thereof as sterilant. These tests have been mainly performed in a STERIZONE® VP4 sterilizer of TSO3 Inc, Quebec (Canada) using a H₂O₂+O₃ sterilization process. Compatibility is evaluated in the number of sterilization cycles to which the endoscope may be sustained before any leak or visible failure is observed.

The replacement of the components, in accordance with the present disclosure, could be performed by the original equipment manufacturer, by an independent repair service organization or by a healthcare center.

Each GIE is a complete system and its compatibility, i.e. lifetime, is limited by their components of lower compatibility since the whole device has to be functional to fulfill its purpose. In other words, when a single component is degraded, typically due to lack of compatibility with an oxidative sterilant, the degradation of the single component reduces the service life of the entire GIE. In order to increase the overall compatibility of a GIE, a plurality of improvements are possible, as described below. Combinations of these improvements are contemplated.

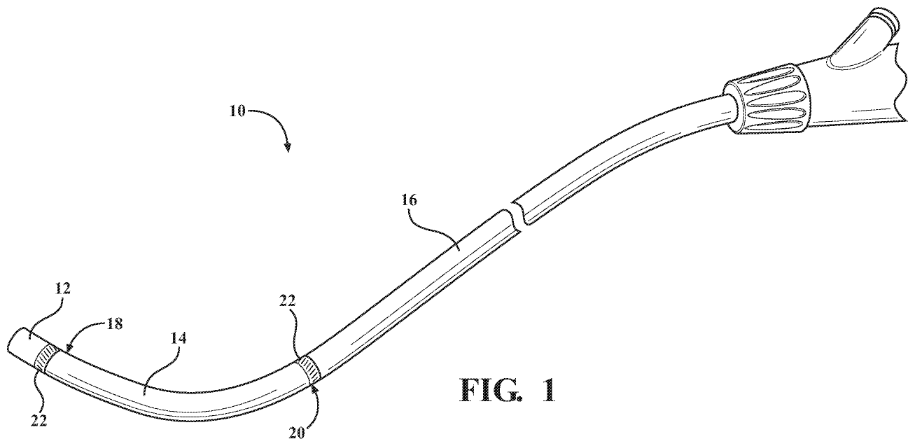

Referring now to FIG. 1, an endoscope 10 includes a distal end region 12. The distal end region 12 may also generally be referred to as a “distal tip”. It is to be appreciated that the distal end region 12 refers to the region that is furthest away from the operator of the endoscope 10. Generally, the distal end region 12 of the endoscope 10 is the most fragile portion of the endoscope 10. Although not required, the distal end region 12 may include various information gathering and functional components. For example, the distal end region 12 of the endoscope 10 may house a light lens assembly, a camera objective, a charge-coupled device (CCD) color chip, and channel openings (e.g. biopsy/suction channels, air/water channel, etc.). The length and diameter of the distal end region 12 will vary depending on the particular application for the endoscope 10 and the amount and size of the various information gathering and functional components included in the distal end region 12.

Referring still to FIG. 1, the endoscope 10 further includes a tubular bending section 14 adjacent the distal end region 12. As the name suggests, the tubular bending section 14 is a flexible tube, which allows the endoscope 10 to maneuver and position the distal end region 12 in the desired direction/orientation. Typically, the tubular bending section 14 includes a central bore extending axially along the tubular bending section 14. Although not required, the tubular bending section 14 typically houses various mechanical components within the central bore, with the mechanical components allowing the tubular bending section 14 to curve or maneuver in any direction. In regards to the composition of the tubular bending section 14, although not required, the tubular bending section 14 is typically formed from an elastomer (e.g. rubber).

As also shown in FIG. 1, the endoscope 10 further includes an insertion tube 16 adjacent the tubular bending section 14 and spaced from the distal end region 12 by the tubular bending section 14. In other words, the tubular bending section 14 links or bridges the distal end region 12 of the endoscope 10 with the insertion tube 16. Similar to the tubular bending section 14, the insertion tube 16 also includes a central bore. Although not required, the insertion tube 16 typically houses various components, such as tubes for suction (biopsy), air, and water feeding, angulation control wires for manipulating the tubular bending section 14, electrical wires connecting the charge-coupled device (CCD) image sensor at the distal end region 12 of the endoscope 10 to a video processor, and glass fibers for bringing light from a light source to the distal end region 12 of the endoscope 10. Suitable endoscope manipulation mechanisms are disclosed in U.S. Pat. No. 10,433,715, U.S. 2016/073855, and U.S. Pat. No. 5,704,898, each of which are hereby incorporated by reference in their respective entirety.

The improvements of the present disclosure increase the compatibility of the endoscope 10 to oxidative processes such as H₂O₂ only sterilization, ozone sterilization, H₂O₂ and ozone sterilization, or H₂O₂+ plasma sterilization as non-limitative examples. Suitable sterilization process are disclosed in U.S. Pat. No. 9,101,679, which is hereby incorporated by reference in its entirety.

Referring still to FIG. 1, the tubular bending section 14 has a first end 18 sealed to distal end region 12 and a second end 20, opposite the first end 18, sealed to the insertion tube 16. In particular, an adhesive element 22 seals the tubular bending section 14 to the distal end region 12 and the insertion tube 16. In other words, the adhesive element 22 forms a fluid-tight bond between the tubular bending section, the distal end region, and the insertion tube 16.

The adhesive element 22 may be an acrylated urethane. Various acrylated urethanes may be use. The acrylated portion of the acrylated urethane may be derived from a methacrylate monomer. In addition, the urethane portion may be derived from a urethane oligomer. For example, the adhesive element 22 is the reaction product of a blend of urethane oligomer and methacrylate monomer. It is to be appreciated that prior to the commencement of the reaction, the actual blend of urethane oligomer and methacrylate monomer may be generally referred to as the adhesive in the uncured state and the adhesive element 22 refers to the cured adhesive. Sealing the connection between the distal end region 12 and the tubular bending section 14 as well as the connection between the tubular bending section 14 and the insertion tube 16 prevents unwanted materials from penetrating into the endoscope 10, and thus protects the functional components housed within the distal end region 12, tubular bending section 14, and insertion tube 16.

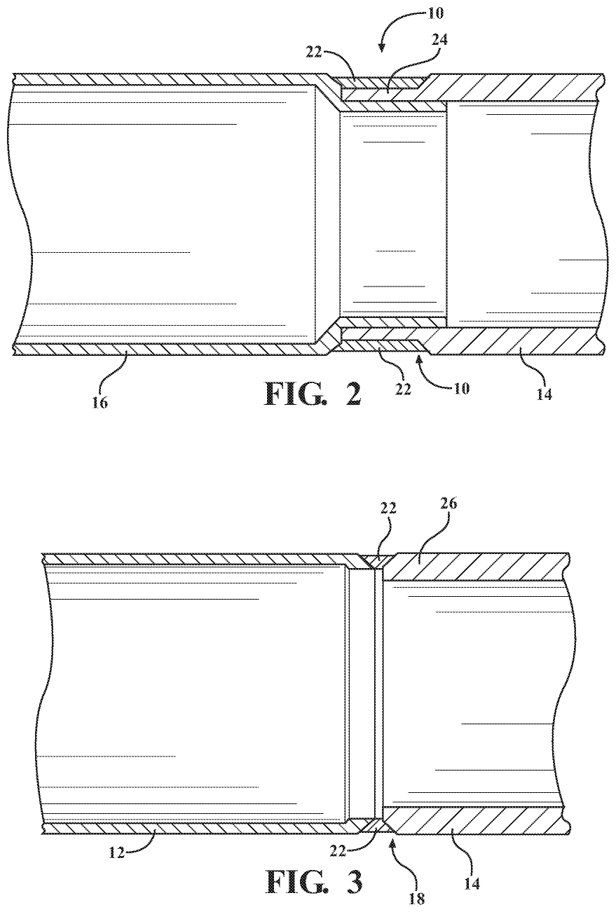

The adhesive element 22 may join the tubular bending section 14 with the distal end region 12 and with the insertion tube 16 by any suitable means. Generally, it is preferred to avoid extending the diameter of the endoscope 10 via the adhesive element 22. Accordingly, as best shown in FIGS. 2 and 3, the first and second ends 18, 20 of the tubular bending section 14 may include tapered lips, 24, 26, with complementary tapered lips on the joining portions of distal end region 12, and insertion tube 16. Utilizing the tapered lips 24 and 26 as a substrate to receive/support the adhesive and adhesive element 22 is a suitable method for sealing the tubular bending section 14 with the distal end region 12 and with the insertion tube 16 without increasing the diameter of the endoscope 10. Other configurations of the ends are also contemplated.

The adhesive may be a one-part UV curable adhesive. The adhesive may optionally have a viscosity of from 3,000 to 15,000 cP, when measured at 20 rpm and in accordance with ASTM D2556. Alternatively, the adhesive may have a viscosity of from 6,000 to 12,000 cP.

The adhesive element 22 may be formed from a plurality of successive adhesive layers, with each successive adhesive layer having a given viscosity lower than a viscosity of a previous adhesive layer. Suitable adhesives include Dymax 1184 MB or Dymax 1184M-V T from Dymax Corp. Each layer may have a different color and/or opacity. For example, a first layer may be transparent and a second layer may be black and opaque.

The present disclosure also provides a method for manufacturing the endoscope 10 with improved compatibility to oxidative processing. The method includes providing the tubular bending section 14 having a first end 18 configured to be sealed to the distal end region 12 and a second end 20 configured to be sealed to the insertion tube 16. The method further includes providing an adhesive comprising a blend of a urethane and an acrylate. The method further includes connecting the first end 18 of the tubular bending section 14 to the distal end region 12 with the adhesive. Finally, the method includes connecting the second end 20 of the tubular bending section 14 to the insertion tube 16 with the adhesive. The method may further include curing the adhesive to form the adhesive element 22. The adhesive may be a blend of urethane oligomer and methacrylate monomer, with the adhesive element 22 being the reaction product thereof.

Connecting the first and second ends 18, 20 may include applying a plurality of successive layers of adhesive, with the adhesive of each successive adhesive layer having a viscosity comparatively lower than a viscosity of the adhesive utilized to form the previous adhesive layer. In such cases, the same adhesive chemistry may be used despite the fact that successive adhesive layers have differing viscosity. Of course, different adhesives (i.e., different adhesive chemistries) may also be used to form the successive layers.

There is also provided a method for repairing an endoscope for enhanced compatibility to oxidative processing. The method includes removing an old (i.e., used) tubular bending section 14 extending between the distal end region 12 and the insertion tube 16. The method further includes providing a new tubular bending section 14 having a first end 18 configured to be sealed to the distal end region 12 and a second end 20 configured to be sealed to the insertion tube 16. The method also includes providing an adhesive comprising a blend of a urethane and an acrylate to connect the first end 18 of the tubular bending section 14 to the distal end region 12 with the adhesive and includes connecting the second end 20 of the tubular bending section 14 to the insertion tube 16 with the adhesive. The method may further include curing the adhesive to form the adhesive element 22 and the endoscope 10. Although not required, the adhesive may be further defined as a blend of urethane oligomer and methacrylate monomer.

### Adhesive at the Distal Cap

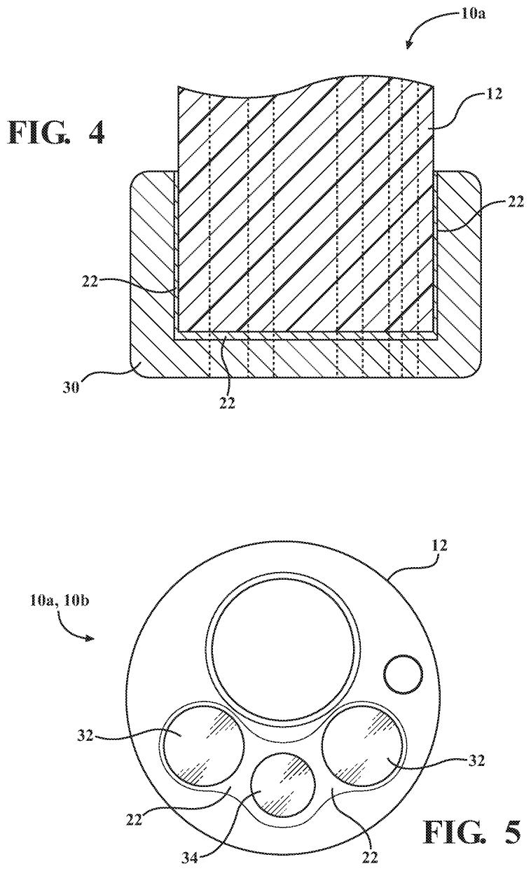

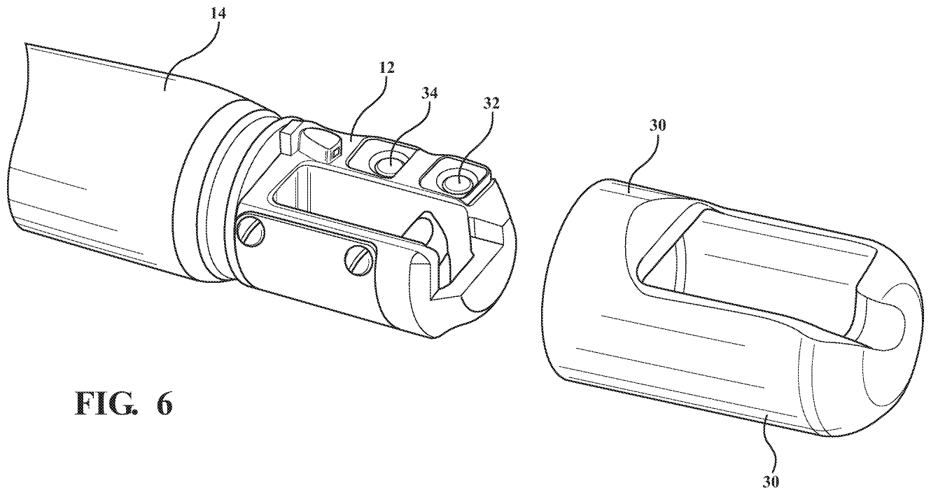

As best shown in FIGS. 4, 6 and 7, the present disclosure also provides an endoscope 10a having a distal cap 30. The distal cap 30 is generally a plastic or polymeric component that covers the distal end region 12 of the endoscope 10a. In particular, the distal cap 30 is bonded to metal included in the distal end region 12 via an adhesive. The bond to metal is desirable to establish superior adhesion to prevent any liquid ingress within the endoscope 10a.

Accordingly, there is provided an endoscope 10a with improved compatibility to oxidative processing. The endoscope 10a having a metallic distal end region 12 and a distal cap 30 bonded to the metallic distal end region 12 via an adhesive element 22. The metallic distal end region 12 may include stainless steel.

The adhesive element 22 may be a cured two-part epoxy. The first-part may include an epoxy and the second-part may include a blend of an amine and an alkyl phenol. The epoxy of the first-part may be an epoxy phenolic novolac and the amine of the second-part may be a polyoxypropylenediamine and the alkyl phenol of the second-part is 4-nonyl phenol.

Alternatively, the two-part epoxy adhesive may contain 60-100% epoxy phenol novolac resins and a mixture of 30-60% of polyoxypropylenediamine and 30-60% 4-nonyl phenol for bonding the distal cap to the metallic distal end region 12. A suitable adhesive is commercially available as EPO-TEK® 302-3M. Typically, the adhesive is a self-cured adhesive configured to cure between 23 to 65° C.

An alternative adhesive element 22 bonding the metallic distal end region 12 to the distal cap 30 may be used. In particular, the adhesive element 22 may be the reaction product of a multifunctional epoxy and an aromatic amine. Although not required, the epoxy may be a poly[(phenyl glycidyl ether)-co-formaldehyde] and the amine may be diethyltoluenediamine. A suitable commercially available adhesive includes Master-Bond EP46HT-2Med.

There is also provided, a method for manufacturing an endoscope 10a with improved compatibility to oxidative processing, the endoscope 10a having the metallic distal end region 12 and the distal cap 30 bonded to the metallic distal end region 12. The method comprises the step of bonding the distal cap 30 to the metallic distal end region 12 with the cured two-part epoxy adhesive or the reaction product of the multifunctional epoxy and the aromatic amine.

There is also provided, a method for repairing an endoscope for enhanced compatibility to oxidative processing. The endoscope has a metallic distal end 12 and an old distal cap bonded to the metallic distal end 12. The method includes removing the old distal cap. The method includes providing a new distal cap 30 and bonding the new distal cap 30 to the metallic distal end region 12 with a two-part epoxy adhesive. Alternatively, the method may include using the reaction product of the multifunctional epoxy and the aromatic amine to bond the new distal cap 30 to the metallic distal end region 12.

### Adhesive Around a Light Guide Lens and a Camera Objective

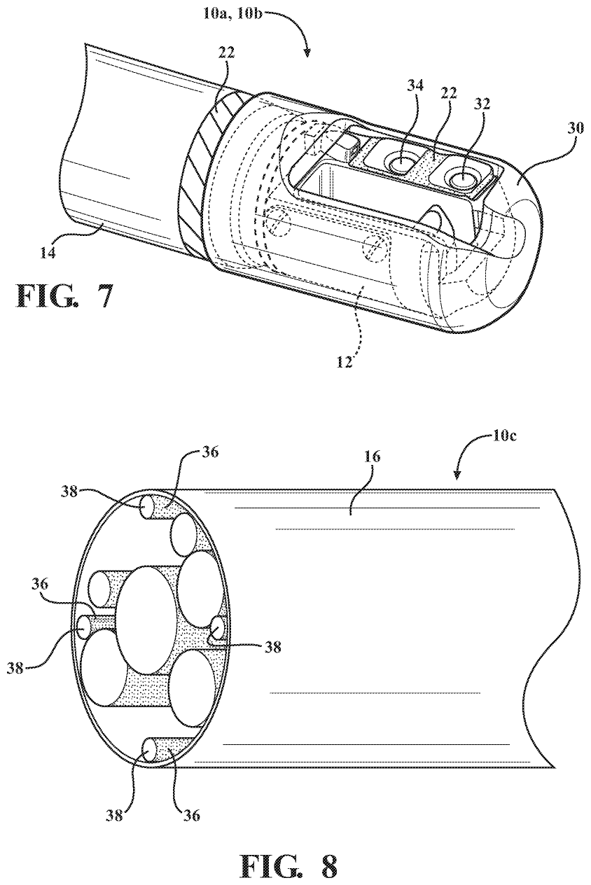

As best shown in FIGS. 5-7, the present disclosure also provides an endoscope 10b having a distal end region 12, a light guide lens 32 and a camera objective 34. The light guide lens 32 and camera objective 34 are optical components integrated with the distal end region 12 of the endoscope 10b. The light guide lens 32 and camera objective 34 are bonded and sealed to the base of the distal end region 12 via adhesive element 22, which secures the light guide lens 32 and camera objective 34 and prevents any liquid ingress into the endoscope 10b. It is to be appreciated that the size of the adhesive element 22 in FIGS. 5 and 7 may be exaggerated for ease of illustration. Four different adhesives used to form the adhesive element 22 and seal the light guide lens 32 and camera objective 34 to the distal end region 12 have been successfully tested. In certain configurations, the light guide lens 32 and the camera objective 34 are on distal face, whereas in other configurations, the light guide lens 32 can be positioned on a lateral face.

Accordingly, there is also provided an endoscope 10b with improved compatibility to oxidative processing, the endoscope 10b having the light guide lens 32 and camera objective 34 sealed to the distal end region 12 with an adhesive element 22.

The adhesive element 22 may be formed from the reaction product of a blend of a urethane and an acrylate. Typically, the reaction product is produced by UV curing the adhesive blend to form the adhesive element 22 in less than 15 minutes. Although not required, the adhesive element 22 may be the reaction product of a blend of urethane oligomer and methacrylate monomer. Suitable adhesives include Dymax 1184 MB or Dymax 1184M-V T from Dymax Corp.

Alternatively, the adhesive element 22 is the reaction product of a two-part epoxy adhesive. The first-part includes an epoxy and the second-part includes a blend of an amine and an alkyl phenol. Typically, the epoxy of the first-part is an epoxy phenolic novolac and the amine of the second-part is a polyoxypropylenediamine and the alkyl phenol of the second-part is 4-nonyl phenol. Although not required, the two-part epoxy adhesive may contain 60-100 wt. % epoxy phenol novolac resins and a mixture of 30-60 wt. % of polyoxypropylenediamine and 30-60 wt. % 4-nonyl phenol for bonding the distal cap 30 to the metallic distal end region 12, with each wt. % based on the total weight of the respective part. In one implementation a suitable adhesive is EPO-TEK® 302-3M from 3M Company. The adhesive may be a self-cured adhesive configured to cure between 23 to 65° C.

The adhesive element 22 may be a cured cyanoacrylate adhesive made of ethyl and octyl cyanoacrylate. In a further configuration, the adhesive is room temperature cured. For example, the adhesive may be medical device adhesive Henkel Loctite 4902.

In another configuration, the adhesive element 22 is an epoxy adhesive. In certain implementations the adhesive element 22 is the reaction product of a multifunctional epoxy and an aromatic amine. Although not required, the epoxy may be a poly[(phenyl glycidyl ether)-co-formaldehyde] and the amine may be diethyltoluenediamine. A suitable adhesive for use in these implementations includes Master-Bond EP46HT-2Med.

In another implementation, the adhesive is a silicone-based adhesive. Suitable adhesives include Nusil MED1.

There is also provided a corresponding method for manufacturing the endoscope 10b with improved compatibility to oxidative processing. The endoscope 10b has the distal end region 12, the light guide lens 32 and the camera objective 34, with each of the light guide lens 32 and the camera objective 34 configured to be sealed to the distal end region 12 with the adhesive element 22. The method comprises applying an adhesive between the light guide lens 32 and the camera objective 34 to the distal end region 12. The method also includes curing the adhesive to form the adhesive element 22. The adhesive is selected from the group consisting of: a blend of a urethane and an acrylate, a two-part epoxy with the first-part comprising an epoxy and the second-part comprising a blend of an amine and an alkyl phenol, a cyanoacrylate, a blend of a multifunctional epoxy and an aromatic amine, and a silicone-based adhesive.

There is also provided a method for repairing an endoscope 10b for enhanced compatibility to oxidative processing. The method includes removing an old (i.e., used) light guide lens and an old camera objective. The method includes providing a new light guide lens 32 and a new camera objective 34, each configured to be sealed to the distal end region 12 with the adhesive element 22. The method comprises the step of applying an adhesive between the new light guide lens 32 and the distal end region 12 and applying the adhesive between the new camera objective 34 and the distal end region 12. The method further includes curing the adhesive to form the adhesive element 22 and seal the new light guide lens 32 and the new camera objective 34. The adhesive is selected from the group consisting of: a blend of a urethane and an acrylate, a two-part epoxy with the first-part comprising an epoxy and the second-part comprising a blend of an amine and an alkyl phenol, a cyanoacrylate, a blend of a multifunctional epoxy and an aromatic amine, a silicone-based adhesive, and combinations thereof.

### Dry Lubricant

A dry lubricant 36 can be changed for a material that fulfill the same mechanical purpose while being compatible to highly oxidative chemistry.

Accordingly, there is provided an endoscope 10c with improved compatibility to oxidative processing, the endoscope 10c having the dry lubricant 36 being a hexagonal boron nitride powder optionally having a granulometry in the range of 1 μm to 20 μm, preferably of 5.0 μm.

Alternatively, the endoscope 10c has a dry lubricant 36 being a fluoropolymer powder. Typically, the fluoropolymer is formed from tetrafluoroethylene units (i.e., monomers). Suitably dry fluoropolymer lubricants include Teflon®.

There is also provided a method for manufacturing an endoscope 10c with improved compatibility to oxidative processing, the method includes the step of lubricating components of the endoscope 10c, the dry lubricant 36 being a hexagonal boron nitride powder having a granulometry in the range of 1 μm to 20 μm, preferably of 5.0 μm.

There is also provided a method for manufacturing the endoscope 10c with improved compatibility to oxidative processing, the method comprising the step of lubricating components of the endoscope 10c using the dry lubricant 36, the dry lubricant 36 being a fluoropolymer powder.

There is also provided a method for repairing an old (i.e., used) endoscope. The method includes providing an old endoscope with an insertion tube 16. The old endoscope includes a plurality of articulation elements 38 disposed within the insertion tube 16, with the articulation elements 38 in contact with an old dry lubricant. The articulation elements 38 are elongated structures that extend through the insertion tube 16 and function to manipulate a tubular bending section 14 of the used endoscope. Suitable examples of the articulation elements 38 include guide wires or guide cables. Although not required, the used endoscope may also include a plurality of channels, electrical wires/cables, optical components, etc. These optional components may also be in contact with the old dry lubricant. The method further includes removing the old dry lubricant with various techniques, such as using mechanical brushing techniques. The method further includes lubricating the plurality of articulation elements 38 with a new dry lubricant 36 to form the endoscope 10c.

The new dry lubricant 36 may be a hexagonal boron nitride powder having a granulometry in the range of 1 μm to 20 μm. Although not required, the granulometry may be about 5.0 μm. Alternatively, the new dry lubricant 36 may be a fluoropolymer powder. Although not required, the fluoropolymer powder may be derived from tetrafluoroethylene units. Typically, the old dry lubricant is molybdenum sulfide.

### Insertion Tube

The outer shell of an insertion tube may be made of elastomeric material resistant to highly oxidative material.

The present disclosure provides an endoscope with improved compatibility to oxidative processing, the endoscope having an insertion tube provided with an outer shell comprising an elastomeric material. The elastomeric material may include a thermoplastic polyurethane coated with a fluoropolymer. A suitable fluoropolymer includes a fluoropolymer derived from tetrafluoroethylene such as Teflon®.

There is also provided a method for manufacturing an endoscope with improved compatibility to oxidative processing, the method comprising the step of using an insertion tube having an outer shell comprising an elastomeric material.

There is also provided a method for repairing an endoscope for enhanced compatibility to oxidative processing, the method comprising using an insertion tube having an outer shell comprising an elastomeric material.

It is further contemplated that any reference to an adhesive element 22 in this disclosure may alternatively include the following formulations as the adhesive element 22. It is also to be appreciated that any reference to a wt. % (within this entire disclosure) of a component in an adhesive or adhesive element 22 is the wt. % based on the total weight of the particular adhesive or adhesive element 22 unless specifically specified to the contrary.

Formula 1: Ethyl 2-cyanoacrylate (30-60 wt. %), Octyl Cyanoacrylate (10-30 wt. %), and thickener (5-10 wt. %). Formula 2: Silanamine, 1,1,1-trimethyl-N-(trimethylsilyl)-, hydrolysis products with silica (10-30 wt. %), siloxanes and Silicones, dimethyl, methyl hydrogen (<20 wt. %). Formula 3: Silanamine, 1,1,1-trimethyl-N-(trimethylsilyl)-, hydrolysis products with silica (20-30 wt. %), Silanetriol, ethyl-, triacetate (<10 wt. %), Glycidoxypropyltrimethoxysilane (<3 wt. %). Formula 4: Silanamine, 1,1,1-trimethyl-N-(trimethylsilyl)-, hydrolysis products with silica (20-40 wt. %), Siloxanes and Silicones, dimethyl, methyl hydrogen (<10 wt. %). Formula 5: ethyl 2-cyanoacrylate (50-100 wt. %), 1,4-dihydroxybenzene (<0.1% wt. %). Formula 6: isobornyl acrylate (25-50 wt. %), N,N-dimethylacrylamide (10-25 wt. %), 3-trimethoxysilylpropyl Methacrylate (2.5-10 wt. %). Formula 7: ethyl 2-cyanoacrylate (50-100 wt. %), 1,4-dihydroxybenzene (<0.1% wt. %). Formula 8, the reaction product of bisphenol-A-(epichlorhydrin) epoxy resin (number average molecular weight <700) (50-99 wt. %), butyl glycidyl ether (1-2.5 wt. %).

While certain methods are described throughout as using a ‘used’ component, it should also be understood that new components may be replaced with other new components in order to utilize the adhesives described throughout to yield an endoscope with improved oxidative processing compatibility.

The following disclosure also provides a number of configurations defined by the following statements.

Statement 1. An endoscope with improved compatibility to an oxidative processing, said endoscope having a distal end and an insertion tube sealingly connected to the distal end, said endoscope comprising:

a tubular bending rubber element having a first end sealed to the distal end and a second end sealed to the insertion tube; and

an adhesive element comprising a blend of urethane oligomer and methacrylate monomer for sealing the tubular bending rubber element.

Statement 2. The endoscope of statement 1, wherein the adhesive element comprises a one-part UV cured adhesive.

Statement 3. The endoscope of any one of statements 1 to 2, wherein the adhesive element has a viscosity comprised between 3000 and 15000 cP.

Statement 4. The endoscope of any one of statements 1 to 3, wherein the adhesive element comprises a plurality of successive layers, each successive layer having a viscosity lower than a viscosity of a previous layer.

Statement 5. The endoscope of any one of statements 1 to 4, wherein the endoscope is a GI endoscope.

Statement 6. The endoscope of any one of statements 1 to 5, wherein the oxidative processing comprises a H₂O₂ sterilization process under vacuum.

Statement 7. The endoscope of any one of statements 1 to 6, wherein the oxidative processing comprises an ozone sterilization process under vacuum.

Statement 8. A method for manufacturing an endoscope with improved compatibility to an oxidative processing, said endoscope having a distal end and an insertion tube configured to be sealingly connected to the distal end, said method comprising the steps of:

providing a tubular bending rubber element having a first end configured to be sealed to the distal end and a second end configured to be sealed to the insertion tube;

providing an adhesive comprising a blend of urethane oligomer and methacrylate monomer;

connecting the first end of the tubular rubber element to the distal end with the adhesive; and

connecting the second end of the tubular rubber element to the insertion tube with the adhesive.

Statement 9. The method of statement 8, wherein the adhesive is a one-part adhesive configured to be UV cured, connecting the first end and the connecting the second end are simultaneously performed, the method further comprising the step of UV curing the adhesive.

Statement 10. The method of any one of statements 8 to 9, wherein providing the adhesive comprises providing an adhesive that has a viscosity comprised between 3000 and 15000 cP.

Statement 11. The method of any one of statements 8 to 10, wherein each of the connecting the first end and the connecting the second end comprises applying a plurality of successive layers of the adhesive, each successive layer having a given viscosity lower than a viscosity of a previous layer.

Statement 12. The method of any one of statements 8 to 11, wherein the endoscope is a GI endoscope.

Statement 13. The method of any one of statements 8 to 12, wherein the oxidative processing comprises a H₂O₂ sterilization process under vacuum.

Statement 14. The method of any one of statements 8 to 13, wherein the oxidative processing comprises an ozone sterilization process under vacuum.

Statement 15. A method for repairing an endoscope for enhanced compatibility to an oxidative processing, said endoscope having a distal end and an insertion tube configures to be sealingly connected to the distal end, said method comprising the steps of:

removing any former tubular bending rubber element extending between the distal end and the insertion tube;

providing a tubular bending rubber element having a first end configured to be sealed to the distal end and a second end configured to be sealed to the insertion tube;

providing an adhesive comprising a blend of urethane oligomer and methacrylate monomer;

connecting the first end of the tubular rubber element to the distal end with the adhesive; and

connecting the second end of the tubular rubber element to the insertion tube with the adhesive.

Statement 16. The method of statement 15, wherein the connecting the first end and the connecting the second end are simultaneously performed, the method further comprising the step of UV curing the adhesive, the adhesive being a one-part adhesive.

Statement 17. The method of any one of statements 15 to 16, wherein the adhesive has a viscosity comprised between 3000 and 15000 cP.

Statement 18. The method of any one of statements 15 to 17, wherein each of the connecting the first end and the connecting the second end comprises applying a plurality of successive layers of the adhesive, each successive layer having a given viscosity lower than a viscosity of a previous layer.

Statement 19. The method of any one of statements 15 to 18, wherein the endoscope is a GI endoscope.

Statement 20. The method of any one of statements 15 to 19, wherein the oxidative processing comprises a H₂O₂ sterilization process under vacuum.

Statement 21. The method of any one of statements 15 to 20, wherein the oxidative processing comprises an ozone sterilization process under vacuum.

### Adhesive at the Distal Cap

Statement 22. An endoscope with improved compatibility to an oxidative processing, said endoscope having a metallic distal end and a distal cap bonded to the metallic distal end, said endoscope comprising: a two-part epoxy adhesive containing 60-100% Epoxy Phenol Novolac Resins and a mixture of 30-60% of Polyoxypropylenediamine and 30-60% 4-nonyl phenol bonding the distal cap to the metallic distal end.

Statement 23. The endoscope of statement 22, wherein the adhesive is a self-cured adhesive configured to cure between 23 to 65° C.

Statement 24. The endoscope of any one of statements 22 to 23, wherein the endoscope is a GI endoscope.

Statement 25. The endoscope of any one of statements 22 to 24, wherein the oxidative processing comprises a H₂O₂ sterilization process under vacuum.

Statement 26. The endoscope of any one of statements 22 to 25, wherein the oxidative processing comprises an ozone sterilization process under vacuum.

Statement 27. An endoscope with improved compatibility to an oxidative processing, said endoscope having a metallic distal end and a distal cap bonded to the metallic distal end, said endoscope comprising: an epoxy adhesive made of 60-100% Poly[(phenyl glycidyl ether)-co-formaldehyde] and 60-100% Diethyltoluenediamine bonding the distal cap to the metallic distal end.

Statement 28. The endoscope of statement 27, wherein the endoscope is a GI endoscope.

Statement 29. The endoscope of any one of statements 27 to 28, wherein the oxidative processing comprises a H₂O₂ sterilization process under vacuum.

Statement 30. The endoscope of any one of statements 27 to 29, wherein the oxidative processing comprises an ozone sterilization process under vacuum.

Statement 31. A method for manufacturing an endoscope with improved compatibility to an oxidative processing, said endoscope having a metallic distal end and a distal cap bonded to the metallic distal end, said method comprising the step of: bonding the distal cap to the metallic distal end with a two-part epoxy adhesive containing 60-100% Epoxy Phenol Novolac Resins and a mixture of 30-60% of Polyoxypropylenediamine and 30-60% 4-nonyl phenol.

Statement 32. The method of statement 31, wherein the adhesive is self-cured between 23 to 65° C.

Statement 33. The method of any one of statements 31 to 32, wherein the endoscope is a GI endoscope.

Statement 34. The method of any one of statements 31 to 33, wherein the oxidative processing comprises a H2O2 sterilization process under vacuum.

Statement 35. The method of any one of statements 31 to 34, wherein the oxidative processing comprises an ozone sterilization process under vacuum.

Statement 36. A method for manufacturing an endoscope with improved compatibility to an oxidative processing, said endoscope having a metallic distal end and a distal cap bonded to the metallic distal end, said method comprising the step of: bonding the distal cap to the metallic distal end with an epoxy adhesive made of 60-100% Poly[(phenyl glycidyl ether)-co-formaldehyde] and 60-100% Diethyltoluenediamine.

Statement 37. The method of statement 36, wherein the endoscope is a GI endoscope.

Statement 38. The method of any one of statements 36 to 37, wherein the oxidative processing comprises a H₂O₂ sterilization process under vacuum.

Statement 39. The method of any one of statements 36 to 38, wherein the oxidative processing comprises an ozone sterilization process under vacuum.

Statement 40. A method for repairing an endoscope for enhanced compatibility to an oxidative processing, said endoscope having a metallic distal end and a distal cap bonded to the metallic distal end, said method comprising the step of:

bonding the distal cap to the metallic distal end with a two-part epoxy adhesive containing 60-100% Epoxy Phenol Novolac Resins and a mixture of 30-60% of Polyoxypropylenediamine and 30-60% 4-nonyl phenol.

Statement 41. The method of statement 40, wherein the adhesive is self-cured between 23 to 65° C.

Statement 42. The method of any one of statements 40 to 41, wherein the endoscope is a GI endoscope.

Statement 43. The method of any one of statements 40 to 42, wherein the oxidative processing comprises a H₂O₂ sterilization process under vacuum.

Statement 44. The method of any one of statements 40 to 43, wherein the oxidative processing comprises an ozone sterilization process under vacuum.

Statement 45. A method for repairing an endoscope for enhanced compatibility to an oxidative processing, said endoscope having a metallic distal end and a distal cap bonded to the metallic distal end, said method comprising the step of: bonding the distal cap to the metallic distal end with an epoxy adhesive made of 60-100% Poly[(phenyl glycidyl ether)-co-formaldehyde] and 60-100% Diethyltoluenediamine.

Statement 46. The method of statement 45, wherein the endoscope is a GI endoscope.

Statement 47. The method of any one of statements 45 to 46, wherein the oxidative processing comprises a H2O2 sterilization process under vacuum.

Statement 48. The method of any one of statements 45 to 47, wherein the oxidative processing comprises an ozone sterilization process under vacuum.

### Adhesive Around the Light Guide Lens and Camera Objective

Statement 49. An endoscope with improved compatibility to an oxidative processing, said endoscope having a distal end, said endoscope further having a light guide lens and a camera objective, each being sealed to the distal end, said endoscope comprising:

an adhesive for sealing each of the light guide lens and the camera objective to the distal end, said adhesive being one selected from a group comprising:

an adhesive comprising a blend of urethane oligomer and methacrylate monomer;

a two-part epoxy adhesive containing 60-100% Epoxy Phenol Novolac Resins and a mixture of 30-60% of Polyoxypropylenediamine and 30-60% 4-nonyl phenol;

a cyanoacrylate adhesive made of ethyl and octyl cyanoacrylate;

an epoxy adhesive made of 60-100% Poly[(phenyl glycidyl ether)-co-formaldehyde] and 60-100% Diethyltoluenediamine; and

a silicone-based adhesive.

Statement 50. The endoscope of statement 49, wherein the adhesive comprising a blend of urethane oligomer and methacrylate monomer is UV cured in less than 15 minutes.

Statement 51. The endoscope of statement 49, wherein the two-part epoxy adhesive is self-cured at 23 to 65° C.

Statement 52. The endoscope of statement 49, wherein the cyanoacrylate adhesive is room temperature cured.

Statement 53. The endoscope of any one of statements 49 to 52, wherein the endoscope is a GI endoscope.

Statement 54. The endoscope of any one of statements 49 to 53, wherein the oxidative processing comprises a H2O2 sterilization process under vacuum.

Statement 55. The endoscope of any one of statements 49 to 54, wherein the oxidative processing comprises an ozone sterilization process under vacuum.

Statement 56. A method for manufacturing an endoscope with improved compatibility to an oxidative processing, said endoscope having a distal end, said endoscope further having a light guide lens and a camera objective, each configured to be sealed to the distal end, said method comprising the step of:

sealing each of the light guide lens and the camera objective to the distal end with an adhesive being one selected from a group comprising:

an adhesive comprising a blend of urethane oligomer and methacrylate monomer;

a two-part epoxy adhesive containing 60-100% Epoxy Phenol Novolac Resins and a mixture of 30-60% of Polyoxypropylenediamine and 30-60% 4-nonyl phenol;

a cyanoacrylate adhesive made of ethyl and octyl cyanoacrylate;

an epoxy adhesive made of 60-100% Poly[(phenyl glycidyl ether)-co-formaldehyde] and 60-100% Diethyltoluenediamine; and

a silicone-based adhesive.

Statement 57. The method of statement 56, wherein the adhesive comprises a blend of urethane oligomer and methacrylate monomer is UV cured in less than 15 minutes.

Statement 58. The method of statement 56, wherein the two-part epoxy adhesive is self-cured at 23 to 65° C.

Statement 59. The method of statement 56, wherein the cyanoacrylate adhesive is room cured.

Statement 60. The method of any one of statements 56 to 59, wherein the endoscope is a GI endoscope.

Statement 61. The method of any one of statements 56 to 60, wherein the oxidative processing comprises a H2O2 sterilization process under vacuum.

Statement 62. The method of any one of statements 56 to 61, wherein the oxidative processing comprises an ozone sterilization process under vacuum.

Statement 63. A method for repairing an endoscope for enhanced compatibility to an oxidative processing, said endoscope having a distal end, said endoscope further having a light guide lens and a camera objective, each configured to be sealed to the distal end, said method comprising the step of:

sealing each of the light guide lens and the camera objective to the distal end with an adhesive being one selected from a group comprising:

an adhesive comprising a blend of urethane oligomer and methacrylate monomer;

a two-part epoxy adhesive containing 60-100% Epoxy Phenol Novolac Resins and a mixture of 30-60% of Polyoxypropylenediamine and 30-60% 4-nonyl phenol;

a cyanoacrylate adhesive made of ethyl and octyl cyanoacrylate;

an epoxy adhesive made of 60-100% Poly[(phenyl glycidyl ether)-co-formaldehyde] and 60-100% Diethyltoluenediamine; and

a silicone-based adhesive.

Statement 64. The method of statement 63, wherein the adhesive comprising a blend of urethane oligomer and methacrylate monomer is UV cured in less than 15 minutes.

Statement 65. The method of statement 63, wherein the two-part epoxy adhesive is self-cured at 23 to 65° C.

Statement 66. The method of statement 63, wherein the cyanoacrylate adhesive is room cured.

Statement 67. The method of any one of statements 63 to 66, wherein the endoscope is a GI endoscope.

Statement 68. The method of any one of statements 63 to 67, wherein the oxidative processing comprises a H2O2 sterilization process under vacuum.

Statement 69. The method of any one of statements 63 to 68, wherein the oxidative processing comprises an ozone sterilization process under vacuum.

### Dry Lubricant

Statement 70. An endoscope with improved compatibility to an oxidative processing, said endoscope comprising a dry lubricant being a hexagonal boron nitride powder having a granulometry of 5.0 μm.

Statement 71. An endoscope with improved compatibility to an oxidative processing, said endoscope comprising a dry lubricant being a fluoropolymer powder.

Statement 72. The endoscope of statement 71, wherein the fluoropolymer comprises tetrafluoroethylene.

Statement 73. The endoscope of any one of statements 70 to 72, wherein the endoscope is a GI endoscope.

Statement 74. The endoscope any one of statements 70 to 73, wherein the oxidative processing comprises a H2O2 sterilization process under vacuum.

Statement 75. The endoscope of any one of statements 70 to 74, wherein the oxidative processing comprises an ozone sterilization process under vacuum.

Statement 76. A method for manufacturing an endoscope with improved compatibility to an oxidative processing, said method comprising the step of lubricating components of the endoscope using a dry lubricant, the dry lubricant being a hexagonal boron nitride powder having a granulometry of 5.0 μm.

Statement 77. A method for manufacturing an endoscope with improved compatibility to an oxidative processing, said method comprising the step of lubricating components of the endoscope using a dry lubricant, the dry lubricant being a fluoropolymer powder.

Statement 78. The method of statement 77, wherein the fluoropolymer comprises tetrafluoroethylene.

Statement 79. The method of any one of statements 76 to 78, wherein the endoscope is a GI endoscope.

Statement 80. The method of any one of statements 76 to 79, wherein the oxidative processing comprises a H₂O₂ sterilization process under vacuum.

Statement 81. The method of any one of statements 76 to 80, wherein the oxidative processing comprises an ozone sterilization process under vacuum.

Statement 82. A method for repairing an endoscope for enhanced compatibility to an oxidative processing, said method comprising: removing existing lubricant; and lubricating components of the endoscope using a dry lubricant, the dry lubricant being a hexagonal boron nitride powder having a granulometry in the range of 1 μm to 20 μm, preferably a granulometry of 5.0 μm.

Statement 83. A method for repairing an endoscope for enhanced compatibility to an oxidative processing, said method comprising: removing existing lubricant; and lubricating components of the endoscope using a dry lubricant, the dry lubricant being a fluoropolymer powder.

Statement 84. The method of statement 83, wherein the fluoropolymer comprises tetrafluoroethylene.

Statement 85. The method of any one of statements 82 to 84, wherein the endoscope is a GI endoscope.

Statement 86. The method of any one of statements 82 to 84, wherein the oxidative processing comprises a H2O2 sterilization process under vacuum.

Statement 87. The method of any one of statements 82 to 84, wherein the oxidative processing comprises an ozone sterilization process under vacuum.

### Insertion Tube

Statement 88. An endoscope with improved compatibility to an oxidative processing, said endoscope comprising: an insertion tube having an outer shell comprising an elastomeric material.

Statement 89. The endoscope of statement 88, wherein the elastomeric material comprises a thermoplastic polyurethane coated with a fluoro polymer.

Statement 90. The endoscope of statement 89, wherein the fluoro polymer comprises tetrafluoroethylene.

Statement 91. The endoscope of any one of statements 88 to 90, wherein the endoscope is a GI endoscope.

Statement 92. The endoscope of any one of statements 88 to 91, wherein the oxidative processing comprises a H2O2 sterilization process under vacuum.

Statement 93. The endoscope of any one of statements 88 to 92, wherein the oxidative processing comprises an ozone sterilization process under vacuum.

Statement 94. A method for manufacturing an endoscope with improved compatibility to an oxidative processing, said method comprising the step of using an insertion tube having an outer shell comprising an elastomeric material.

Statement 95. The method of statement 94, wherein the elastomeric material comprises a thermoplastic polyurethane coated with a fluoro polymer.

Statement 96. The method of statement 95, wherein the fluoro polymer comprises tetrafluoroethylene.

Statement 97. The method of any one of statements 94 to 96, wherein the endoscope is a GI endoscope.

Statement 98. The method of any one of statements 94 to 97, wherein the oxidative processing comprises a H2O2 sterilization process under vacuum.

Statement 99. The method of any one of statements 94 to 98, wherein the oxidative processing comprises an ozone sterilization process under vacuum.

Statement 100. A method for repairing an endoscope for enhanced compatibility to an oxidative processing, said method comprising the step of using an insertion tube having an outer shell comprising an elastomeric material.

Statement 101. The method of statement 100, wherein the elastomeric material comprises a thermoplastic polyurethane coated with a fluoro polymer.

Statement 102. The method of statement 101, wherein the fluoro polymer comprises tetrafluoroethylene.

Statement 103. The method of any one of statements 100 to 102, wherein the endoscope is a GI endoscope.

Statement 104. The method of any one of statements 100 to 103, wherein the oxidative processing comprises a H2O2 sterilization process under vacuum.

Statement 105. The method of any one of statements 100 to 104, wherein the oxidative processing comprises an ozone sterilization process under vacuum.

Although the above description relates to specific preferred embodiments as presently contemplated by the inventors, it will be understood that the invention in its broad aspect includes mechanical and functional equivalents of the elements described herein.

1. An endoscope with improved compatibility to oxidative processing, said endoscope having a distal end region, said endoscope comprising
an insertion tube sealingly connected to the distal end region
a tubular bending section having a first end sealed to the distal end region and a second end sealed to the insertion tube; and
an adhesive element comprising an acrylated urethane, said adhesive element sealing the tubular bending section to the distal end region and to the insertion tube.

2. The endoscope of claim 1, wherein the adhesive element is a reaction product of a urethane oligomer and an acrylate.

3. The endoscope of claim 2, wherein the acrylate is a methacrylate monomer.

4. The endoscope of claim 2, wherein prior to curing the adhesive element a blend of the urethane oligomer and the acrylate has a viscosity of from 3000 to 15000 cP, when measured at 20 rpm and in accordance with ASTM D2556.

5. The endoscope of claim 1, wherein the endoscope is a GI endoscope.

6. A method for manufacturing an endoscope with improved compatibility to oxidative processing, said endoscope having a distal end region and an insertion tube configured to be sealingly connected to the distal end region, said method comprising the steps of:
providing a tubular bending section having a first end configured to be sealed to the distal end region and a second end configured to be sealed to the insertion tube;
providing an adhesive comprising a blend of urethane and acrylate;
connecting the first end of the tubular bending section to the distal end region with the adhesive; and
connecting the second end of the tubular bending section to the insertion tube with the adhesive.

7. The method of claim 6, wherein the adhesive is a one-part adhesive configured to be UV cured, the method further comprising the step of UV curing the adhesive.

8. The method of claim 6, wherein providing the adhesive comprises providing an adhesive that has a viscosity of from 3000 to 15000 cP, when measured at 20 rpm and in accordance with ASTM D2556.

9. The method of claim 6, wherein the urethane is a urethane oligomer and the acrylate is a methacrylate monomer.

10. The method of claim 6, wherein connecting the first end and the connecting the second end individually comprise applying a plurality of successive layers of the adhesive, with each successive layer having a viscosity lower than a viscosity of a previous layer.

11. The method of claim 6, wherein the endoscope is a GI endoscope.

12. The method of claim 6, further comprising sterilizing the endoscope in a H₂O₂ sterilization process under vacuum, an ozone sterilization process under vacuum, or a H₂O₂ and ozone sterilization process under vacuum.

13. A method for repairing an endoscope, the endoscope having a distal end region and an insertion tube configured to be sealingly connected to the distal end region, said method comprising the steps of:
removing an old tubular bending section extending between the distal end region and the insertion tube;
providing a new tubular bending section having a first end configured to be sealed to the distal end region and a second end configured to be sealed to the insertion tube;
providing an adhesive comprising a blend of a urethane and an acrylate monomer;
connecting the first end of the tubular bending section to the distal end region with the adhesive;
connecting the second end of the tubular bending section to the insertion tube with the adhesive; and
UV curing the adhesive;
wherein the endoscope has enhanced compatibility with a H₂O₂ sterilization process under vacuum, an ozone sterilization process under vacuum, or a H₂O₂ and ozone sterilization process under vacuum.

14. The method of claim 13, wherein the urethane is a urethane oligomer and the acrylate is a methacrylate monomer.

15. The method of claim 13, wherein the blend of urethane and acrylate has a viscosity of from 3,000 to 15,000 cP, when measured at 20 rpm and in accordance with ASTM D2556.

16.-53. (canceled)
